(19) United States
(12) Patent Application Publication
(10) Pub. No.: US 2018/0022670 A1
(43) Pub. Date: Jan. 25, 2018

(54) METHOD FOR EMULSION REMOVAL IN AMINE REMOVAL UNIT

(71) Applicant: SABIC Global Technologies B.V., Bergen op Zoom (NL)

(72) Inventors: Bander Bawareth, Riyadh (SA); Shahid Azam, Riyadh (SA); Patel Haresh, Jubail (SA); Deshmukh V. Rajan, Jubail (SA); Fahad Mubark Al-Khaldi, Jubail (SA); Muhamad Rafiq Bn-Hasan Attamim, Jubail (SA)

(21) Appl. No.: 15/544,093

(22) PCT Filed: Jan. 15, 2016
(86) PCT No.: PCT/IB2016/050196

§ 371 Date: Jul. 17, 2017

Related U.S. Application Data
(60) Provisional application No. 62/104,341, filed on Jan. 16, 2015.

(51) Int. Cl. C07C 7/10; B01D 11/04

(52) U.S. Cl. CPC C07C 7/10; B01D 11/0492; B01D 11/0488

(57) ABSTRACT

A method for removal of an organic amine from a hydrocarbon stream includes mixing the hydrocarbon stream including the amine with an aqueous inorganic acid in a volumetric ratio of hydrocarbon stream: aqueous inorganic acid of greater than 1.5:1 to 5:1, phase separating a hydrocarbon phase and an aqueous phase, removing the hydrocarbon phase, mixing the aqueous phase with an aqueous alkaline solution, phase separating the aqueous phase and an organic phase, and removing the organic phase. The aqueous inorganic acid has a concentration of greater than or equal to 30 weight percent. The disclosed method results in improved phase separation time during an amine removal process.

### BACKGROUND

In the chemical industry, processes are often conducted resulting in an outlet stream product or a feed stream to a process unit comprising hydrocarbon and amines An example is the outlet stream from a reactor used for preparing linear alpha olefins (LAOS) by oligomerization of ethylene. The linear alpha olefin products are then separated into different fractions for further use or marketing. Often, an amine is added during the oligomerization process or is added into a reactor outlet piping system.

In many cases, it is difficult to remove the organic amine from the hydrocarbon stream by distillation, because the boiling points of the amine and the hydrocarbon stream (or fractions thereof) are very close. For example, n-dodecylamine (DDA) is often added to oligomerization processes, which after product fractionation is carried through to the C₁₄ linear alpha olefin product fraction. Since DDA has a boiling point close to the C₁₄ linear alpha olefin product, it cannot readily be removed by distillation. Similarly, 2-ethylhexylamine (2-EHA) has a very close boiling point to the C₁₀ linear alpha olefin product fraction, and cannot be easily removed by distillation.

Alternative methods for removal of an organic amine from a hydrocarbon stream can include the steps of reacting the amine of the hydrocarbon stream containing the amine with an acid, extracting the amine salt formed into an aqueous phase, and optionally recovering and recycling the amine However, emulsions frequently result from this process, and phase separation of organic and aqueous phases using this method is time consuming. The plant can be forced to shut down, which is detrimental to industrial processes.

There is an active interest in overcoming the above-described technical limitations, to more efficiently remove an amine from a hydrocarbon stream, and to avoid emulsion formation during the recovery process. Accordingly, there remains a need for an improved method of removing an amine from a hydrocarbon stream using phase separation.

### BRIEF DESCRIPTION

A method for removal of an organic amine from a hydrocarbon stream comprises: mixing the hydrocarbon stream comprising the amine with an aqueous inorganic acid in a volumetric ratio of hydrocarbon stream: aqueous inorganic acid of 1.5:1 to 5:1, phase separating a hydrocarbon phase and an aqueous phase, removing the hydrocarbon phase, mixing the aqueous phase with an aqueous alkaline solution, phase separating the aqueous phase and an organic phase, and removing the organic phase, wherein the aqueous inorganic acid has a concentration of greater than or equal to 30 weight percent (wt. %).

A method for removal of an organic amine from a hydrocarbon stream comprises: mixing the hydrocarbon stream comprising the amine with an aqueous inorganic acid in a volumetric ratio of hydrocarbon stream: aqueous inorganic acid of 2:1 to 4:1, phase separating a hydrocarbon phase and an aqueous phase, removing the hydrocarbon phase, mixing the aqueous phase with an aqueous alkaline solution, phase separating the aqueous phase and an organic phase, and removing the organic phase, wherein the aqueous inorganic acid has a concentration of 30 to 40 wt. %, wherein the hydrocarbon stream is a C₁₀ linear alpha olefin product fraction, wherein the organic amine is 2-ethylhexylamine, and wherein the aqueous inorganic acid is aqueous hydrochloric acid.

These and other features and characteristics are more particularly described below.

### BRIEF DESCRIPTION OF THE DRAWINGS

The following is a brief description of the drawing wherein like elements are numbered alike and which are presented for the purposes of illustrating the exemplary embodiments disclosed herein and not for the purposes of limiting the same.

FIG. 1 illustrates a schematic representation of the emulsion removal process in an amine removal unit.

### DETAILED DESCRIPTION

Described herein is a method for removal of an organic amine from a hydrocarbon stream wherein the phase separation time of an emulsion is reduced. It was unexpectedly discovered that the existing feed to the amine removal unit could be manipulated to break an emulsion in the amine removal unit, thus precluding the use of any additional chemicals. The use of an aqueous acid having a specified concentration and used in a specific volume ratio relative to the organic phase can provide improved phase separation and optimized amine recovery and removal.

A method for removal of an organic amine from a hydrocarbon stream can include mixing the hydrocarbon stream comprising the amine with an aqueous inorganic acid in a volumetric ratio of hydrocarbon stream: aqueous inorganic acid of, for example, greater than 1.5:1 to 5:1. A hydrocarbon phase and an aqueous phase can be phase separated and the hydrocarbon phase removed. The aqueous phase can be mixed with an aqueous alkaline solution and the aqueous phase and an organic phase can be phase separated. The organic phase can be removed. The aqueous inorganic acid can have a concentration of greater than or equal to 30 weight percent (wt. %).

A method for removal of an organic amine from a hydrocarbon stream can include mixing the hydrocarbon stream with an aqueous inorganic acid in a volumetric ratio of hydrocarbon stream: aqueous inorganic acid of 2:1 to 4:1. The hydrocarbon stream can include the amine A hydrocarbon phase and an aqueous phase can be phase separated and the hydrocarbon phase can be removed. The aqueous phase can be mixed with an aqueous alkaline solution and the aqueous phase and the organic phase can be phase separated. The organic phase can then be removed. The aqueous inorganic acid can have a concentration of 30 to 40 wt. %. The hydrocarbon stream can include a C₁₀ to C₁₈ linear alpha olefin product fraction, for example, a C₁₀ linear alpha olefin product fraction. The organic amine can include 2-ethylhexylamine and the aqueous inorganic acid can be aqueous hydrochloric acid.

The hydrocarbon stream can be an outlet stream or a fraction thereof from a reactor for preparing linear alpha olefins by oligomerization of an olefin. The linear alpha olefins are generally addition products containing greater than or equal to two ethylene units, but not as many ethylene units as in the relatively high molecular weight addition product referred to as polyethylene. The hydrocarbon stream can be a fraction of an outlet stream, for example a product fraction comprising C₁₀ to C₁₈ linear alpha olefins. For example, the fraction can be a C₁₀ linear alpha olefin product fraction.

Accordingly, the method for emulsion removal in an amine removal unit disclosed herein can desirably be included in a method for preparing linear alpha olefins by oligomerization of ethylene, for example, in the presence of a solvent and a catalyst. Oligomerization of ethylene can be conducted according to any method that is generally known, for example, the method can comprise the steps of feeding ethylene into an oligomerization reactor, oligomerizing the ethylene in the reactor, and removing an outlet stream comprising linear alpha olefins from the reactor via a reactor outlet piping system.

An organic amine can be added into an oligomerization reaction and/or into the reactor outlet piping system during a process for preparing linear alpha olefins. The organic amine can be a primary, secondary, tertiary, or cyclic amine. The organic amine can be soluble in an organic phase containing linear alpha olefins (e.g., the hydrocarbon stream). The organic amine can also be insoluble or have low solubility in an aqueous phase. For example, the organic amine can be 2-ethylhexylamine, n-dodecylamine, n-decylamine, tributylamine, trihexylamine, 3-ethylheptylamine, t-butylamine, triethylamine, cyclopentylamine, t-octylamine, n-heptylamine, 2-heptylamine, hexylamine, dihexylamine, 1,6-diaminohexane, tributylamine, 1,8-diaminooctane, tris-2-ethylhexylamine, or a combination thereof. In one embodiment, the organic amine is 2-ethylhexylamine

The aqueous inorganic acid can include aqueous hydrogen halides (e.g., hydrochloric acid, hydrobromic acid, and hydrofluoric acid), aqueous boric acid, aqueous nitric acid, aqueous phosphoric acid, and aqueous sulfuric acid. For example, the aqueous inorganic acid can be aqueous hydrochloric acid. The aqueous inorganic acid can have a concentration of greater than or equal to 30 wt. %, for example, 30 to 40 wt. %, for example, 30 wt. %. For example, the aqueous inorganic acid can be aqueous hydrochloric acid having a concentration of 30 wt. %.

The phase separation time of an emulsion formed in an amine removal unit can be optimized by adjusting the volumetric ratio of hydrocarbon stream to aqueous inorganic acid. The volumetric ratio of hydrocarbon stream to aqueous inorganic acid can be greater than 1.5:1 to 5:1, for example 2:1 to 4:1. Use of this volumetric ratio can improve phase separation, which is beneficial to processing time and associated costs. For example, phase separation of the hydrocarbon phase and the aqueous phase can occur in less than or equal to 60 seconds, for example, less than or equal to 45 seconds, for example, less than or equal to 35 seconds. Time to achieve phase separation can be reduced by greater than or equal to 50%, for example, greater than or equal to 60%, for example, greater than or equal to 70%, as compared to a process not using a volumetric ratio of hydrocarbon stream to aqueous inorganic acid of greater than 1.5:1 to 5:1.

Following phase separation, the hydrocarbon phase containing the purified linear alpha olefin product (e.g., 1-decene) can be removed by decanting. The hydrocarbon phase can optionally be further purified. For example, at least a part of the hydrocarbon phase can be recycled into the step of mixing the hydrocarbon stream and the inorganic acid. Further purification can also comprise washing the hydrocarbon phase with water and passing the hydrocarbon phase through an absorbing agent, for example, silica gel, alumina, molecular sieves, and the like.

The remaining aqueous phase can then be mixed with an aqueous alkaline solution, neutralizing the amine salt to recover the organic amine The aqueous alkaline solution can be, for example, aqueous sodium hydroxide (NaOH), potassium hydroxide (KOH), calcium hydroxide (Ca(OH)₂), or any other aqueous alkaline media, and can have a concentration of 1 to 30 wt. %, for example, 10 to 30 wt. %, for example 15 to 25 wt. %, for example, 20 wt. %. The organic amine can be selected so as to have only limited solubility in water, as previously described herein. The neutralized organic amine can phase separate from the aqueous phase to form a separate organic phase which can subsequently be removed, for example, by decanting. The organic phase containing the organic amine can be further purified and can be, for example, washed with water one or more times to minimize the amount of residual acid and/or alkaline, followed by subsequent water removal steps, for example, distillation. The purified organic amine can be recycled, for example, for addition to linear alpha olefin reactor outlet lines or into an oligomerization reactor.

Advantageously, the cost associated with use of the organic amine in the production of linear alpha olefins can be considerably reduced because the amine can be purified, recovered, and recycled by the method disclosed herein. An organic amine recovered by the method disclosed herein can be stored, for example, in an amine storage unit, and recycled into an oligomerization reaction and/or into a reactor outlet piping system.

Various mixing elements, for example, static or dynamic mixers, can be utilized for optimizing the reaction efficiency in any of the steps of the method disclosed herein. Furthermore, the method steps (mixing with aqueous acid, aqueous alkaline or washing with water) can be performed in a once-through mode, or the reaction/washing efficiencies can be enhanced by installation of cycles.

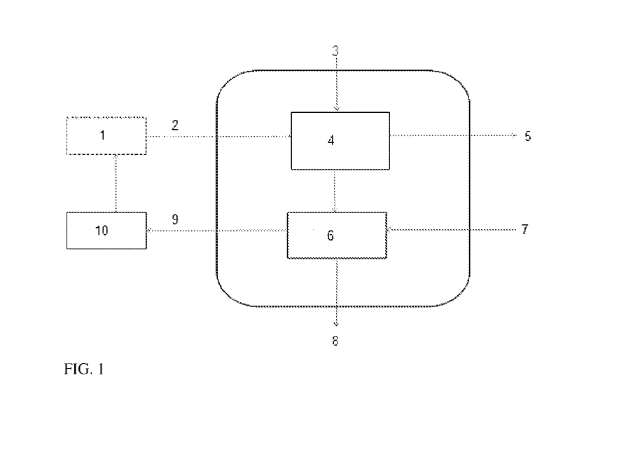

A method for removing an amine from a hydrocarbon stream can be carried out according to the process depicted in FIG. 1. A hydrocarbon stream can be an outlet stream from a reactor (1) for preparing linear alpha olefins. The outlet stream (2) can be a product fraction, for example a crude C₁₀ product fraction comprising an organic amine (e.g., 2-ethylhexylamine). The product fraction (2) can be transferred to an acid decanter (4), where an aqueous inorganic acid (e.g., 30 wt. % HCl) (3) can be injected and mixed with the organic hydrocarbon stream. Upon injecting the aqueous acid, the organic amine can be protonated to form an amine salt, for example, according to the following:

1-decene+2-EHA+HCl→2-EHA.HCl+1-decene

The amine salt is soluble in the aqueous phase, and can therefore be extracted from the hydrocarbon phase into the aqueous phase. Following phase separation, the purified hydrocarbon phase can be removed by decanting (5). The aqueous phase including the amine salt can be transferred to a second decanter (6), where the amine salt can be neutralized by the addition of an aqueous alkaline solution (7), for example 20 wt. % sodium hydroxide (NaOH). Neutralization can allow for recovery of the organic amine according to the following:

2-EHA.HCl+NaOH+2-EHA+H₂O+NaCl

The recovered organic amine can form an organic phase, and can be extracted from the aqueous phase. The aqueous salt waste can be removed (8) and the recovered amine (9) can be transferred to amine storage (10). The recovered organic amine can optionally be recycled into the linear alpha olefin reactor (1).

The method disclosed herein can provide a method for reducing or eliminating an emulsion during amine removal. Manipulating the feed to control the volumetric ratio of hydrocarbon stream to aqueous inorganic acid can reduce or eliminate emulsion formation in the amine removal unit without requiring additional chemicals. Therefore, a substantial improvement in amine removal from linear alpha olefin product streams can be provided.

The following example is merely illustrative of the method disclosed herein and is not intended to limit the scope hereof.

### EXAMPLE

In the following example, a hydrocarbon stream comprising a C₁₀ product fraction was mixed with an aqueous inorganic acid according to the volume ratios provided in Table 1. The aqueous inorganic acid was HCl in a concentration of 30 wt. %. The hydrocarbon stream and the inorganic acid were mixed in a first decanter. The time for phase separation to occur was measured in seconds (s). As can be seen from Table 1, improved separation time was achieved when a volumetric ratio of hydrocarbon stream to aqueous inorganic acid of greater than 1.5:1 was used.

TABLE 1
Phase separation time at various hydrocarbon: aqueous ratios

| | Concentration of | Volume ratio | Separation |
|---|---|---|---|
| | EHA salt and | (Hydrocarbon: | time |
| Example # | HCl in acid cycle | Aqueous) | (s) |
| 1 | EHA-Cl: 14.4 wt. % | 70:30 | 30/31/32 |
| | HCl: 1.3 wt.% | | |
| Comparative | EHA-Cl: 9.0 wt. % | 60:40 | 120/110/115 |
| Example 2 | HCl: 7.2 wt. % | | |

As illustrated by Example 1, an amine removal process according to the method disclosed herein using a volumetric ratio of hydrocarbon stream to aqueous inorganic acid of 2.3:1 resulted in improved phase separation times. The average phase separation time over three runs was 31 seconds. Comparative Example 2 demonstrated that the average phase separation time for a volumetric ratio of hydrocarbon stream to aqueous inorganic acid of 1.5:1 was 115 seconds. Thus, the time to achieve phase separation was reduced by about 73% when a volumetric ratio of 2.3:1 was used, as in Example 1. The data in Table 1 demonstrates that phase separation of a hydrocarbon phase and an aqueous phase can be significantly improved by the addition of aqueous HCl having a concentration of 30 wt. % when used in a volumetric ratio of hydrocarbon stream to aqueous inorganic acid of greater than 1.5:1.

The method for removal of an organic amine from a hydrocarbon stream disclosed herein include at least the following embodiments:

Embodiment 1: A method for removal of an organic amine from a hydrocarbon stream, comprising mixing the hydrocarbon stream comprising the amine with an aqueous inorganic acid in a volumetric ratio of hydrocarbon stream: aqueous inorganic acid of greater than 1.5:1 to 5:1; phase separating a hydrocarbon phase and an aqueous phase; removing the hydrocarbon phase; mixing the aqueous phase with an aqueous alkaline solution; phase separating the aqueous phase and an organic phase; and removing the organic phase; wherein the aqueous inorganic acid has a concentration of greater than or equal to 30 wt. %.

Embodiment 2: The method of Embodiment 1, wherein phase separating the hydrocarbon phase and the aqueous phase occurs in less than or equal to 60 seconds.

Embodiment 3: The method of Embodiments 1 or 2, wherein phase separating the hydrocarbon phase and the aqueous phase occurs in less than or equal to 45 seconds.

Embodiment 4: The method of any of Embodiments 1 to 3, wherein phase separating the hydrocarbon phase and the aqueous phase occurs in less than or equal to 35 seconds.

Embodiment 5: The method of any one of Embodiments 1 to 4, wherein the hydrocarbon stream comprising the amine is an outlet stream or a fraction thereof from a reactor for preparing linear alpha olefins by oligomerization of an olefin.

Embodiment 6: The method of Embodiment 5, wherein the fraction is a product fraction comprising C₁₀ to C₁₈ linear alpha olefins.

Embodiment 7: The method of Embodiments 5 or 6, wherein the fraction is a C₁₀ linear alpha olefin product fraction.

Embodiment 8: The method of any of Embodiments 1 to 7, wherein the organic amine is 2-ethylhexylamine

Embodiment 9: The method of any of Embodiments 1 to 8, wherein the aqueous inorganic acid is aqueous hydrochloric acid.

Embodiment 10: The method of any of Embodiments 1 to 9, wherein the aqueous inorganic acid has a concentration of 30 to 40 wt. %.

Embodiment 11: The method of any of Embodiments 1 to 10, wherein the aqueous inorganic acid has a concentration of 30 wt. %.

Embodiment 12: The method of any of Embodiments 1 to 11, further comprising decanting to remove the hydrocarbon phase.

Embodiment 13: The method of any of Embodiments 1 to 12, wherein the volumetric ratio of hydrocarbon stream: aqueous inorganic acid is 2:1 to 4:1.

Embodiment 14: The method of any of Embodiments 1 to 13, wherein the aqueous alkaline solution is aqueous sodium hydroxide.

Embodiment 15: The method of any of Embodiments 1 to 14, wherein the aqueous alkaline solution has a concentration of 20 wt. %.

Embodiment 16: The method of any of Embodiments 1 to 15, further comprising recycling at least a part of the hydrocarbon phase into the step of mixing the hydrocarbon stream comprising the amine with an aqueous inorganic acid.

Embodiment 17: The method of any of Embodiments 1 to 16, further comprising further purifying the hydrocarbon phase, wherein further purification comprises washing the hydrocarbon phase with water and passing the hydrocarbon phase through an absorbing agent.

Embodiment 18: The method of any of Embodiments 1 to 17, further comprising further purifying the organic phase, wherein further purification comprises washing the organic phase with water, removing the aqueous phase, and distilling any residual water from the organic phase.

Embodiment 19: A method for removal of an organic amine from a hydrocarbon stream, comprising mixing the hydrocarbon stream comprising the amine with an aqueous inorganic acid in a volumetric ratio of hydrocarbon stream: aqueous inorganic acid of 2:1 to 4:1; phase separating a hydrocarbon phase and an aqueous phase; removing the hydrocarbon phase; mixing the aqueous phase with an aqueous alkaline solution; phase separating the aqueous phase and an organic phase; and removing the organic phase; wherein the aqueous inorganic acid has a concentration of 30 to 40 wt. %; wherein the hydrocarbon stream is a C₁₀ linear alpha olefin product fraction; wherein the organic amine is 2-ethylhexylamine; and wherein the aqueous inorganic acid is aqueous hydrochloric acid.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs. A “combination” is inclusive of blends, mixtures, alloys, reaction products, and the like.

As used herein, the term “hydrocarbyl” and “hydrocarbon” refers broadly to a substituent comprising carbon and hydrogen, optionally with 1 to 3 heteroatoms, for example, oxygen, nitrogen, halogen, silicon, sulfur, or a combination thereof; “alkyl” refers to a straight or branched chain, saturated monovalent hydrocarbon group; “alkylene” refers to a straight or branched chain, saturated, divalent hydrocarbon group; “alkylidene” refers to a straight or branched chain, saturated divalent hydrocarbon group, with both valences on a single common carbon atom; “alkenyl” refers to a straight or branched chain monovalent hydrocarbon group having at least two carbons joined by a carbon-carbon double bond; “cycloalkyl” refers to a non-aromatic monovalent monocyclic or multicyclic hydrocarbon group having at least three carbon atoms, “cycloalkenyl” refers to a non-aromatic cyclic divalent hydrocarbon group having at least three carbon atoms, with at least one degree of unsaturation; “aryl” refers to an aromatic monovalent group containing only carbon in the aromatic ring or rings; “arylene” refers to an aromatic divalent group containing only carbon in the aromatic ring or rings; “alkylaryl” refers to an aryl group that has been substituted with an alkyl group as defined above, with 4-methylphenyl being an exemplary alkylaryl group; “arylalkyl” refers to an alkyl group that has been substituted with an aryl group as defined above, with benzyl being an exemplary arylalkyl group; “acyl” refers to an alkyl group as defined above with the indicated number of carbon atoms attached through a carbonyl carbon bridge (—C(═O)—); “alkoxy” refers to an alkyl group as defined above with the indicated number of carbon atoms attached through an oxygen bridge (—O—); and “aryloxy” refers to an aryl group as defined above with the indicated number of carbon atoms attached through an oxygen bridge (—O—).

Unless otherwise indicated, each of the foregoing groups can be unsubstituted or substituted, provided that the substitution does not significantly adversely affect synthesis, stability, or use of the compound. The term “substituted” as used herein means that at least one hydrogen on the designated atom or group is replaced with another group, provided that the designated atom's normal valence is not exceeded. When the substituent is oxo (i.e., ═O), then two hydrogens on the atom are replaced. Combinations of substituents and/or variables are permissible provided that the substitutions do not significantly adversely affect synthesis or use of the compound. Exemplary groups that can be present on a “substituted” position include, but are not limited to, cyano; hydroxyl; nitro; azido; alkanoyl (such as a C₂₋₆ alkanoyl group such as acyl); carboxamido; C₁₋₆ or C₁₋₃ alkyl, cycloalkyl, alkenyl, and alkynyl (including groups having at least one unsaturated linkages and from 2 to 8, or 2 to 6 carbon atoms); C₁₋₆ or C₁₋₃ alkoxyl; C₆₋₁₀ aryloxy such as phenoxy; C₁₋₆ alkylthio; C₁₋₆ or C₁-₃ alkylsulfinyl; C1-6 or C₁₋₃ alkylsulfonyl; aminodi(C₁₋₆ or C₁₋₃)alkyl; C₆₋₁₂ aryl having at least one aromatic rings (e.g., phenyl, biphenyl, naphthyl, or the like, each ring either substituted or unsubstituted aromatic); C₇₋₁₉ arylalkyl having 1 to 3 separate or fused rings and from 6 to 18 ring carbon atoms; or arylalkoxy having 1 to 3 separate or fused rings and from 6 to 18 ring carbon atoms, with benzyloxy being an exemplary arylalkoxy.

All cited patents, patent applications, and other references are incorporated herein by reference in their entirety. However, if a term in the present application contradicts or conflicts with a term in the incorporated reference, the term from the present application takes precedence over the conflicting term from the incorporated reference

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

I/we claim:

1. A method for removal of an organic amine from a hydrocarbon stream, comprising:
mixing the hydrocarbon stream comprising the amine with an aqueous inorganic acid in a volumetric ratio of hydrocarbon stream: aqueous inorganic acid of greater than 1.5:1 to 5:1;
phase separating a hydrocarbon phase and an aqueous phase;
removing the hydrocarbon phase;
mixing the aqueous phase with an aqueous alkaline solution;
phase separating the aqueous phase and an organic phase; and
removing the organic phase;
wherein the aqueous inorganic acid has a concentration of greater than or equal to 30 wt. %.

2. The method of claim 1, wherein phase separating the hydrocarbon phase and the aqueous phase occurs in less than or equal to 60 seconds.

3. The method of claim 1, wherein phase separating the hydrocarbon phase and the aqueous phase occurs in less than or equal to 45 seconds.

4. The method of claim 1, wherein phase separating the hydrocarbon phase and the aqueous phase occurs in less than or equal to 35 seconds.

5. The method of claim 1, wherein the hydrocarbon stream comprising the amine is an outlet stream or a fraction thereof from a reactor for preparing linear alpha olefins by oligomerization of an olefin.

6. The method of claim 5, wherein the fraction is a product fraction comprising C₁₀ to C₁₈ linear alpha olefins.

7. The method of claim 5, wherein the fraction is a C₁₀ linear alpha olefin product fraction.

8. The method of claim 1, wherein the organic amine is 2-ethylhexylamine.

9. The method of claim 1, wherein the aqueous inorganic acid is aqueous hydrochloric acid.

10. The method of claim 1, wherein the aqueous inorganic acid has a concentration of 30 to 40 wt. %.

11. The method of claim 1, wherein the aqueous inorganic acid has a concentration of 30 wt. %.

12. The method of claim 1, further comprising decanting to remove the hydrocarbon phase.

13. The method of claim 1, wherein the volumetric ratio of hydrocarbon stream: aqueous inorganic acid is 2:1 to 4:1.

14. The method of claim 1, wherein the aqueous alkaline solution is aqueous sodium hydroxide.

15. The method of claim 1, wherein the aqueous alkaline solution has a concentration of 20 wt. %.

16. The method of claim 1, further comprising recycling at least a part of the hydrocarbon phase into the step of mixing the hydrocarbon stream comprising the amine with an aqueous inorganic acid.

17. The method of claim 1, further comprising further purifying the hydrocarbon phase, wherein further purification comprises washing the hydrocarbon phase with water and passing the hydrocarbon phase through an absorbing agent.

18. The method of claim 1, further comprising further purifying the organic phase, wherein further purification comprises washing the organic phase with water, removing the aqueous phase, and distilling any residual water from the organic phase.

19. A method for removal of an organic amine from a hydrocarbon stream, comprising:
mixing the hydrocarbon stream comprising the amine with an aqueous inorganic acid in a volumetric ratio of hydrocarbon stream: aqueous inorganic acid of 2:1 to 4:1;
phase separating a hydrocarbon phase and an aqueous phase;
removing the hydrocarbon phase;
mixing the aqueous phase with an aqueous alkaline solution;
phase separating the aqueous phase and an organic phase; and
removing the organic phase;
wherein the aqueous inorganic acid has a concentration of 30 to 40 wt. %;
wherein the hydrocarbon stream is a Cio linear alpha olefin product fraction;
wherein the organic amine is 2-ethylhexylamine; and
wherein the aqueous inorganic acid is aqueous hydrochloric acid.
